(19) United States
(12) Patent Application Publication
(10) Pub. No.: US 2010/0160751 A1
(43) Pub. Date: Jun. 24, 2010

(54) PHOTOPLETHYSMOGRAPHIC SENSOR BASED BLOOD GAS MONITOR DEVICE FOR ANALYSIS, RESEARCH AND CALIBRATION IN AN EXTRACORPOREAL CIRCUIT OR EXTRACORPOREAL PULSE SIMULATION SYSTEM

(75) Inventors: Bernard F. Hete, Kittanning, PA (US); Eric W. Starr, Allison Park, PA (US); Eric J. Ayers, Aliquippa, PA (US)

Correspondence Address: BLYNN L. SHIDELER;THE BLK LAW GROUP, 3500 BROKKTREE ROAD, SUITE 200, WEXFORD, PA, 15090, US

(73) Assignee: STARR Life Sciences Corp., Oakmont, PA (US)

(21) Appl. No.: 12/704,024

(22) Filed: Feb. 11, 2010

Related U.S. Application Data
(63) Continuation of application No. PCT/US08/73926, filed on Aug. 21, 2008.
(60) Provisional application No. 60/956,955, filed on Aug. 21, 2007.
(60) Provisional application No. 61/029,081, filed on Feb. 15, 2008.

(51) Int. Cl. A61B 5/1455

(52) U.S. Cl. 600/322

(57) ABSTRACT

A blood oxygenation monitoring device may comprise an extracorporeal pulse simulation system including one at least partially transparent blood holding element with a photoplethysmographic sensor coupled to the element and adapted to measure particular gas content of the blood. The system includes a pulse simulation mechanism configured to simulate pulsatile behavior of the blood within the element relative to the photoplethysmographic sensors. The blood holding element may be a reservoir, wherein the pulse simulation mechanism includes a magnetic stirrer and stir bar within the reservoir. The blood holding member may be flexible tubing having blood flow there through, wherein the pulse simulation mechanism is a peristaltic pump coupled to the tubing. The monitoring device can rapidly and accurately form oxygen dissociation curves. The monitoring device can be utilized in conjunction with a heart lung bypass machine or other extra corporeal circuit devices or can be a calibration tool for sensors.

### RELATED APPLICATIONS

The present invention is a continuation of International patent application PCT/US08/73926 filed Aug. 21, 2008 which published Feb. 26, 2009 as WO2009-026,468, which is incorporated herein by reference. International patent application PCT/US08/73926 claims the benefit of Provisional Patent application Ser. No. 60/956,955 filed on Aug. 21, 2007 and Provisional Patent application Ser. No. 61/029,081 filed on Feb. 15, 2008.

### BACKGROUND OF THE INVENTION

1. Field of the Invention

The present invention relates to blood gas monitoring and more particularly to a photoplethysmographic sensor based blood oxygenation monitoring in an extracorporeal circuit or an extracorporeal pulse simulation system.

2. Background Information

The present invention relates to blood gas monitoring. The following definitions will be helpful in explaining the known background elements that are helpful for understanding the present invention.

As background, “blood” is a highly specialized circulating tissue consisting of several types of cells suspended in a fluid medium known as plasma. The cellular constituents are: red blood cells (erythrocytes), which carry respiratory gases and give it its red color because they contain hemoglobin (an iron-containing protein that binds oxygen in the lungs and transports it to tissues in the body), white blood cells (leukocytes), which fight disease, and platelets (thrombocytes), cell fragments which play an important part in the clotting of the blood.

Hemoglobin, also spelled haemoglobin and abbreviated Hb, is the iron-containing oxygen-transport metalloprotein in the red blood cells of the blood. In mammals the protein makes up about 97% of the red cell's dry content, and around 35% of the total content (including water). Hemoglobin transports oxygen from the lungs to the rest of the body where it releases its load of oxygen. The name hemoglobin is the concatenation of heme and globin, reflecting the fact that each subunit of hemoglobin is a globular protein with an embedded heme (or haem) group; each heme group contains an iron atom, and this is responsible for the binding of oxygen. The most common type of hemoglobin in mammals contains four such subunits, each with one heme group. In humans, each heme group is able to bind one oxygen molecule, and thus, one hemoglobin molecule can bind four oxygen molecules.

A plethysmograph is an instrument for measuring changes in volume within a body (usually resulting from fluctuations in the amount of blood or air it contains).

A photoplethysmograph is an optically obtained plethysmograph, a volumetric measurement of an organ. A photoplethysmograph is often obtained by using a pulse oximeter which illuminates the skin and measures changes in light absorption A conventional pulse oximeter monitors the perfusion of blood to the dermis and subcutaneous tissue of the skin.

A pulse oximeter is a medical device that indirectly measures the amount of a gas, typically oxygen in a patient's blood, which is opposed to measuring oxygen saturation directly through a blood sample, and changes in blood volume in the skin, producing a photoplethysmogragh. It is generally attached to a medical monitor to display the results such as constant oxygen saturation. The construction and operation of pulse oximeters are known in the art.

Photoplethysmograph Pulse Oximetry Measurements reference a class or family of known calculations used to determine the pulse and oxygenation measurements of a subject. Photoplethysmograph Pulse Oximetry Measurements require a pulsetile behavior in the associated subject in order to obtain caclulations. Photoplethysmograph Co-Oximetry Measurements reference a class or family of known calculations used to determine the oxygenation measurements of a subject, which does not require pulsetile behavior in the subject. Photoplethysmograph Co-Oximetry Measurements does require measurements from at least two wavelengths of light. Photoplethysmograph Oximetry Measurements is a generic term covering both Photoplethysmograph Pulse Oximetry Measurements and Photoplethysmograph Co-Oximetry Measurements, among others.

The phrase “partially transparent” within the meaning of this application will mean that the material is transparent to at least a plurality of wavelengths of light commonly utilized within photoplethysmograph pulse oximeters. In optics, transparency is the property of allowing light to pass. Though transparency usually refers to visible light in common usage, it may correctly be used, as here, to refer to any type of radiation.

The oxygen dissociation curve is a graph that shows the percent saturation of hemoglobin at various partial pressures of oxygen. Commonly a curve may be expressed with the P₅₀ value. This is a value which tells the pressure at which the erythrocytes are fifty percent saturated with oxygen. The purpose of an oxygen dissociation curve is to show the equilibrium of oxyhemoglobin and nonbonded hemoglobin at various partial pressures. At high partial pressures of oxygen, usually in the lungs, hemoglobin binds to oxygen to form oxyhemoglobin. When the blood is fully saturated all the erythrocytes are in the form of oxyhemoglobin. As the erythrocytes travel to tissues deprived of oxygen the partial pressure of oxygen will decrease. Consequently, the oxyhemoglobin releases the oxygen to form hemoglobin. The sigmoid shape of the oxygen dissociation curve is a result of the cooperative binding of oxygen to the four polypeptide chains. Cooperative binding is the characteristic of hemoglobin having a greater ability to bind oxygen after a subunit has bound oxygen. Thus, hemoglobin is most attracted to oxygen when three of the four polypeptide chains are bound to oxygen.

An extracorporeal medical procedure is a medical procedure which is carried outside the body. It is usually a procedure in which blood is taken from a patient's circulation to have a process applied to it before it is returned to the circulation. All of the apparatus carrying the blood outside the body is termed the extracorporeal circuit. Some definitions of “extracorporeal circuit” require the circuit to be continuous with the bodily circulation, however, within the meaning of this application it will reference the broader meaning of a blood carrying circuit outside of the body.

The phrase extracorporeal pulse simulator system within the meaning of this application will reference a blood containing system outside of the body that includes a mechanism to simulate a pulse in the system. The extracorporeal circuits described herein can also be extracorporeal pulse simulator systems provided the systems include a pulse simulation mechanism. The extracorporeal pulse simulator system within the meaning of this application are not limited to extracorporeal circuits, as the blood containing system may not form a circuit but may have a pulse simulation mechanism.

A Heart Lung Machine, also known as a Pump-Oxygenator or Cardiopulmonary Bypass Machine, is a machine that temporarily takes over the function of the heart and lungs during surgery. It maintains the circulation of blood and the oxygen content of the body. The principle of the heart-lung machine is actually quite simple. Blue blood withdrawn from the upper heart chambers is drained (by gravity siphon) into a reservoir. From there, the blood is pumped through an artificial lung. This component is designed to expose the blood to oxygen. As the blood passes through the artificial lung (also known as an oxygenator), the blood comes into intimate contact with the fine surfaces of the device itself. Oxygen gas is delivered to the interface between the blood and the device, permitting the blood cells to absorb oxygen molecules directly. Now the blood is red in color, indicating its rich content of oxygen destined to be delivered to the various tissues of the body. Finally, the heart-lung machine actively pumps the red blood back into the patient through a tube connected to the arterial circulation. The heart-lung circuit is a continuous loop; as the red blood goes into the body, blue blood returns from the body and is drained into the pump completing the circuit. The modern heart-lung machine is actually more sophisticated and versatile than the overview given above.

In fact, the pump-oxygenator can do a number of other tasks necessary for safe completion of an open heart operation. Firstly, any blood which escapes the circulation and spills into the operating field around the heart can be suctioned and returned to the pump. This scavenging feature is made possible because the blood has been rendered incapable of clotting by large doses of heparin. Returning shed blood into the heart-lung machine greatly preserves the patients own blood stores throughout the operation. Secondly, the patient's body temperature can be controlled by selectively cooling or heating the blood as it moves through the heart-lung machine. Thus the surgeon can use low body temperatures as a tool to preserve the function of the heart and other vital organs during the period of artificial circulation. And the bypass pump has connectors into which medications and anesthetic drugs can be given. In this way, medications arrive to the patient almost instantly by simply adding them to the blood within the heart-lung reservoir.

In hemodialysis, the patient's blood is pumped through the blood compartment of a dialyzer, exposing it to a semipermeable membrane. Dialysis solution is pumped through the dialysate compartment of the dialyzer, which is configured so that the blood and dialysis solutions flow on opposite sides of the semipermeable membrane. The cleansed blood is then returned via the circuit back to the body. Ultrafiltration occurs by increasing the hydrostatic pressure across the dialyzer membrane. This usually is done by applying a negative pressure to the dialysate compartment of the dialyzer. This pressure gradient causes water and dissolved solutes to move from blood to dialysate, and allows removal of several liters of excess salt and water during a typical 3-4 hour treatment. Dialysis patient weight is measured in kilos: therefore, one kilo of fluid equals 2.2 pounds of body weight. Hemodialysis treatments are typically given three times per week, but more frequent sessions, which are usually 2-3 hours in duration given 5-6 times per week can be sometimes prescribed. Hemodialysis treatments can be given either in outpatient dialysis centers or can be done by a patient at home, providing they have suitable help and accommodation.

Hemofiltration is a similar treatment to hemodialysis, but it makes use of a different principle. The blood is pumped through a dialyzer or “hemofilter” as in dialysis, but no dialysate is used. A pressure gradient is applied; as a result, water moves across the very permeable membrane rapidly, facilitating the transport of dissolved substances, importantly ones with large molecular weights, which are cleared less well by hemodialysis. Salts and water lost from the blood during this process are replaced with a “substitution fluid” that is infused into the extracorporeal circuit during the treatment. Hemodiafiltration is a term used to describe several methods of combining hemodialysis and hemofiltration in one process.

Plasmapheresis is the removal, treatment, and return of (components of) blood plasma from blood circulation. During plasmapheresis, blood is initially taken out of the body through a needle or previously implanted catheter. Plasma is then removed from the blood by a cell separator. Three procedures are commonly used to separate the plasma from the blood: Discontinuous flow centrifugation—One venous catheter line is required. Typically, a 300 ml batch of blood is removed at a time and centrifuged to separate plasma from blood cells. Continuous flow centrifugation—Two venous lines are used. This method requires slightly less blood volume to be out of the body at any one time as it is able to continuously spin out plasma. Plasma filtration—Two venous lines are used. The plasma is filtered using standard hemodialysis equipment. This continuous process requires less than 100 ml of blood to be outside the body at one time. Each method has its advantages and disadvantages. After plasma separation, the blood cells are returned to the person undergoing treatment, while the plasma, which contains the antibodies, is first treated and then returned to the patient in traditional plasmapheresis.

Apheresis is a medical technology in which the blood of a donor or patient is passed through an apparatus that separates out one particular constituent and returns the remainder to the circulation.

In intensive care medicine, extracorporeal membrane oxygenation (ECMO) is an extracorporeal technique of providing both cardiac and respiratory support oxegen to patients whose heart and lungs are so severely diseased that they can no longer serve their function. An ECMO machine is similar to a heart lung machine.

A peristaltic pump is a type of positive displacement pump used for pumping a variety of fluid. The fluid is contained within a flexible tube generally fitted inside a circular pump casing (though linear peristaltic pumps have been made). In a circular pump a rotor with a number of ‘rollers’, ‘shoes’ or ‘wipers’ attached to the external circumference compresses the flexible tube. As the rotor turns, the part of tube under compression closes (or ‘occludes’) thus forcing the fluid to be pumped to move through the tube. Additionally, as the tube opens to its natural state after the passing of the cam (‘restitution’) fluid flow is induced to the pump. This process is called peristalsis and is used in many biological systems.

A magnetic stirrer is a type of laboratory equipment consisting of a rotating magnet, or stationary electomagnets, creating a rotating magnetic field. The stirrer is used to cause a stir bar, also called a flea, immersed in a liquid to be stirred, to spin very quickly, stirring it. Stirrers are often used in laboratories and are preferred over gear-driven motorized stirrers in chemical research because they are quieter, more efficient, and have no moving parts to break or wear out (other than the simple bar magnet itself). Due to the small size, the stirring bar is more easily cleaned and sterilized than other stirring devices. Mr. Rosinger obtained an early magnetic stirrer patent, U.S. Pat. No. 2,350,534, incorporated herein by reference, which includes a description of a coated bar magnet placed in a vessel, which is driven by a rotating magnet in a base below the vessel. The patent explains that coating the magnet in plastic or covering it with glass or porcelain makes it chemically inert. An even earlier U.S. patent for a magnetic mixer is U.S. Pat. No. 1,242,493, incorporated herein by reference, to Mr. Stringham discloses an early magnetic mixer used stationary electromagnets in the base, rather than a rotating permanent magnet, to rotate the stirrer.

The stir bar, or flea, is the magnetic bar, used to stir a mixture in a vessel. The stir bar rotates (and thus stirs) in synch with a separate rotating magnet located beneath the vessel containing the mixture. Glass, and plastic, does not affect a magnetic appreciably (it is transparent to magnetism) and most chemical reactions take place in glass vessels. This allows magnetic stir bars to work well in glass and plastic vessels. The plastic-coated bar magnet was allegedly independently invented in the late 1940's by Mr. McLaughlin, who named it the ‘flea’ because of the way it jumps about if the rotating magnet is driven too fast.

U.S. publication 2007-0123787 was cited in the international search report of the parent application as a “document defining the general state of the art which is not considered to be of particular relevance.” This reference describes a “pulse wave data analyzing method for extracting vital information out of pulse wave data concerning a living body. The method comprises a noise removal step of: detecting bottom values and peak values along a time axis in a time-series manner out of pulse wave data obtained by sequentially measuring a pulse wave of a subject for a predetermined period; making pairs with respect to the bottom values and the peak values adjacent to each other on the time axis to obtain bottom-to-peak amplitude values along the time axis, the bottom-to-peak amplitude value being a difference between the bottom value and the peak value in each of the pairs; and comparing each set of the two bottom-to-peak amplitude values adjacent to each other along the time axis to remove the bottom value and the peak value relating to the smaller bottom-to-peak amplitude value in the each set as a noise, if a ratio of the one of the two bottom-to-peak amplitude values to the other one of the two bottom-to-peak amplitude values is larger than a predetermined value.”

U.S. publication 2007-0129645 was cited in the international search report of the parent application as a “document defining the general state of the art which is not considered to be of particular relevance.” This reference describes systems “and methods provide for determining blood gas saturation based on one or more measured respiration parameters. A parameter of respiration is measured implantably over a duration of time. The measured respiratory parameter is associated with a blood gas saturation level. Blood gas saturation is determined based on the measured respiration parameter. At least one of associating the measured respiratory parameter and determining blood gas saturation is preferably preformed implantably.”

U.S. publication 2004-0127800 was cited in the international search report of the parent application as a “document defining the general state of the art which is not considered to be of particular relevance.” This reference describes a device which “is provided for assessing impairment of blood circulation in a patient, such as that in perfusion failure, by measurement of blood flow adjacent a mucosal surface accessible by a mouth or nose and connecting with the gastrointestinal tract or upper respiratory/digestive tract of a patient. The device includes a blood-flow sensor adapted to be positioned adjacent a mucosal surface with a patient's body and measuring blood flow in adjacent tissue and a PCO₂ sensor adapted to be positioned adjacent the mucosal surface and measuring PCO₂. In addition a pH sensor may be used in combination with the blood flow determination. A method of detecting perfusion failure is also disclosed. The method includes utilizing blood-flow measurements in conjunction with a surface perfusion pressure index and/or an optical plethysmography index to more accurately assess perfusion failure. These measurements may also be supplement by taking measurements of pH, sublingual PCO₂, and Sa O₂. The invention affords rapid measurement and detection of perfusion failure.”

U.S. publication 2007-0118027 was cited in the international search report of the parent application as a “document defining the general state of the art which is not considered to be of particular relevance.” This reference describes “systems, devices, and/or methods for assessing body fluid-related metrics and/or changes therein. The disclosure further provides systems, devices, and/or methods for correlating body fluid-related metrics in a particular tissue with the corresponding whole-body metric. The disclosure also provides, systems, devices, and/or methods for assessment of such metrics to facilitate diagnosis and/or therapeutic interventions related to maintaining and/or restoring body fluid balance.”

There remains a need in the art to for a simple to operate, intuitive, accurate blood gas monitoring devices for extracorporeal circuits and in extracorporeal pulse simulation system.

### SUMMARY OF THE INVENTION

Some of the above objects are achieved with the blood oxygenation monitoring device according to the present invention that comprises an extracorporeal pulse simulation system wherein the extracorporeal pulse simulation system includes one at least partially transparent blood holding element with a photoplethysmographic sensor coupled to the blood holding element and adapted to measure particular gas content of the blood within the element. The extracorporeal pulse simulation system further includes a pulse simulation mechanism configured to simulate pulsatile behavior of the blood within the element relative to the photoplethysmographic sensors

In one non-limiting embodiment of the present invention the blood holding element is a reservoir and wherein the pulse simulation mechanism includes a magnetic stirrer and a stir bar within the reservoir. In another non-limiting embodiment of the present invention the extracorporeal pulse simulation system is an extracorporeal circuit and the blood holding element is an at least partially transparent flexible tubing having blood flow therethrough, and wherein the pulse simulation mechanism is a peristaltic pump coupled to the tubing and adapted to have blood flow therethrough in a pulsatile manner.

Some of the above objects are achieved with the blood oxygenation monitoring device according to the present invention that comprises an at least partially transparent blood holding reservoir; a photoplethysmographic sensor coupled to the blood holding reservoir and adapted to measure particular gas content of the blood within the reservoir; and a pulse simulation mechanism configured to simulate pulsatile behavior of the blood within the reservoir relative to the photoplethysmographic sensors.

Some of the above objects are achieved with the blood oxygenation monitoring device according to the present invention that comprises an at least partially transparent flexible tubing having blood flow therethrough, a peristaltic pump coupled to the tubing and adapted to have blood flow therethrough in a pulsetile manner, and a photoplethysmographic pulse oximeter sensor coupled to the flexible tube and adapted to measure oxygen content of the blood within the tubing.

The monitoring device can be utilized in an extracorporeal circuit to rapidly and accurately form oxygen dissociation curves. The monitoring device can be utilized in conjunction with existing extracorporeal circuits, such as a heart lung bypass machine, a machine for hemodialysis, a machine for hemofiltration, a machine for plasmapheresis, a machine for apheresis, or a machine for extracorporeal membrane oxygenation, to precisely measure the oxygenation amounts of supplied blood. The monitoring device can be utilized as a calibration tool for sensors such as pulse oximeters. The use of different sensors will allow the device to be used to monitor different blood gases such as carbon monoxide.

These and other advantages of the present invention will be clarified in the description of the preferred embodiments taken together with the attached drawings in which like reference numerals represent like elements throughout.

These and other advantages of the present invention will be clarified in the brief description of the preferred embodiment taken together with the drawings in which like reference numerals represent like elements throughout.

### BRIEF DESCRIPTION OF THE DRAWINGS

FIG. 1 is an overview schematic view of a blood oxygenation monitoring device according to one embodiment of the present invention, such as may be used to rapidly and accurately form oxygen dissociation curves and other blood gas analysis;

FIG. 2 is a schematic section view of one structure for minimizing receipt of ambient light in the sensor of the present invention;

FIG. 3 is an overview schematic view of a blood oxygenation monitoring device according to one embodiment of the present invention used with an existing extracorporeal circuit to precisely measure the oxygenation amounts of supplied blood;

FIG. 4 is an overview schematic view of a blood oxygenation monitoring device according to one embodiment of the present invention used for calibration of blood gas sensors;

FIG. 5 is an overview schematic view of a blood oxygenation monitoring device according to the present invention, such as may be used to rapidly and accurately form oxygen dissociation curves and other blood gas analysis; and

FIG. 6 is an enlarged schematic view of the blood oxygenation monitoring device of FIG. 5.

### DESCRIPTION OF THE PREFERRED EMBODIMENTS

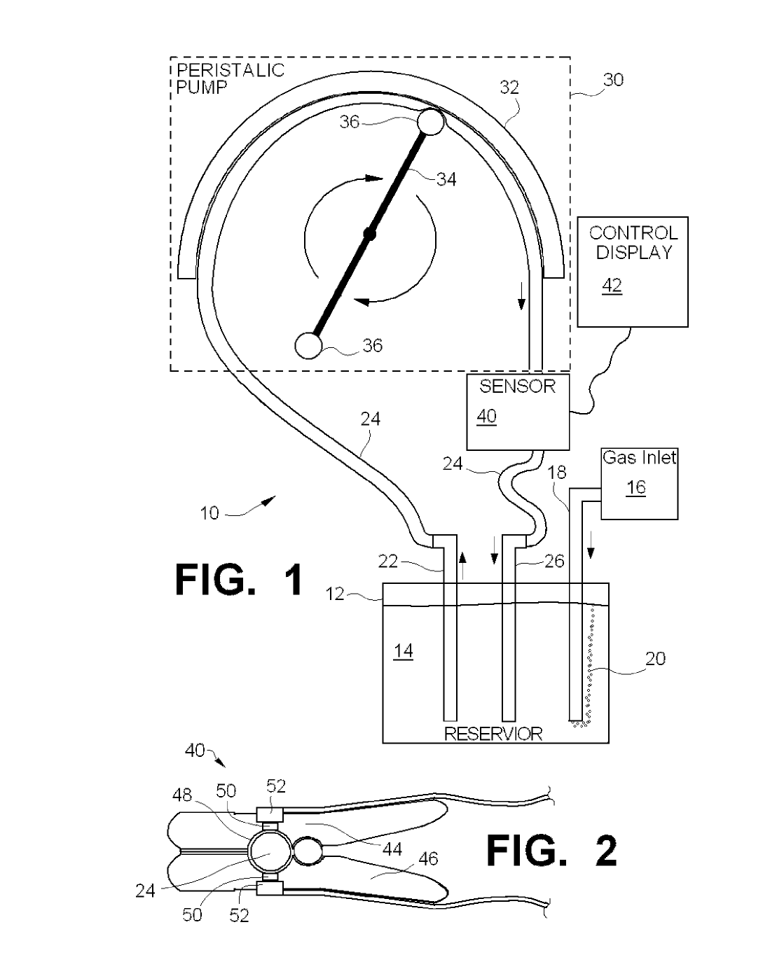

FIG. 1 is an overview schematic view of a blood oxygenation monitoring device 10 according to the present invention, such as may be used to rapidly and accurately form oxygen dissociation curves, as one representative example, and other blood gas analysis in the extracorporeal circuit.

The device 10 includes a reservoir 12 for holding blood 14 that is to be analyzed with the device 10. The device 10 may further include a gas source or inlet 16 attached to coupling 18 that is configured to supplying given amounts of a designated gas 20, e.g. oxygen or carbon monoxide, into the blood 14 within the reservoir 12.

The device 10 has the blood 14 flow through an extracorporeal circuit through an outlet coupling 22 that is coupled to flexible tubing 24 that returns to an inlet coupling 26 to the reservoir 12. The flexible tubing 24 is conventional surgical tubing which is partially transparent within the meaning of the present invention.

A pulse simulation mechanism 30, in the form of a peristaltic pump 30 in the initial embodiment, is used for pumping the blood 14 through the circuit. The blood 14 is contained within the flexible tube 24 fitted inside a circular pump casing 32. A rotor 34 with a number of ‘rollers’, ‘shoes’ or ‘wipers’ 36 attached to the external circumference compresses the flexible tube 24 during rotation of the rotor 34. As the rotor 34 turns, the part of tube 24 under compression closes (or ‘occludes’) thus forcing the blood 14 to be pumped to move through the tube 24. The pump 30 may be replaced with a linear peristaltic pump or other pump resulting in pulsatile flow of the blood. A critical feature of the device 10 is that the flow of blood through the tube 24 be provided by the pump 30 in measurable, volumetric pulses that are detectable with photoplethysmographic sensors, such as 40, using Photoplethysmograph Pulse Oximetry Measurements. In this manner the pump 30 acts as a pulse simulation mechanism for the device 10.

The device according to the present invention provides at least one conventional photoplethysmographic sensor 40 onto the tube 24, generally downstream of the pump 30. The sensor 40 will be associated with a display unit 42 that can record and/or display the measured results. Any conventional photoplethysmographic sensor 40 can be utilized in the device 10, such as the Mouse Ox™ brand device from Starr Life Sciences, or devices from Nellcor or Massimo or other well know providers of photoplethysmographic sensors. It is important that the pump rate for the pump 30 be within an acceptable range for simulating a pulse that is appropriate for the associated sensor 40 using Photoplethysmograph Pulse Oximetry Measurements. The pump rate as relevant to pulse simulation would be equal, essentially to the RPM of the rotor 34 multiplied by the number of rollers 36. The Mouse Ox™ brand device from Starr Life Sciences generally has a higher acceptable pulse range than other conventional sensors used for larger mammals such as humans. The pump rate of the illustrated circuit is generally analogous to the heart rate that the sensors 40 are ordinarily intended to measure. If the pump rate is outside of an acceptable range for the sensor 40 then no meaningful measurements may be obtained, for example the internal signal processing may inadvertently cut off (filter out) the portion of the obtained signal that is actually the signal of interest. Various sensors will generally provide the associated acceptable ranges in the product literature.

An alternative embodiment of the present invention is to utilize Photoplethysmograph Co-Oximetry Measurements with the sensor 40. In this embodiment it may be preferable that the tubing 24 not be flexible in the area of the sensor 40 so that the tubing 24 can be more easily factored out in the associated analysis of the blood. The flexible tubing, such as surgical tubing, is believed to beneficial where pulsetile behavior is provided and where Photoplethysmograph Pulse Oximetry Measurements are utilized.

FIG. 2 is a schematic section view of structure for sensor 40 for minimizing receipt of ambient light in the sensor 40 in accordance with one aspect of the present invention. The sensor 40 is a transmissive sensor as shown with an upper half 44 and a lower half 46 forming a pivoted spring biased clip. The clip includes openings 50 adjacent a conventional transmitter and receiver pair 52. The clip further includes tube receiving grooves 48 adjacent respective openings 50. The grooves 48 and openings 50 allow for the easy transmission and receipt of the appropriate signals. The clip can be made opaque, non-transparent, to limit the amount of ambient light that is received by the receiver 52, distorting the signal of interest. The pivoted clip structure, in general, is a well known pulse oximetry sensor applicator for attaching the pulse oximeter to the finger or earlobe of a patient. The use of grooves 48 is similar to a small mammal pulse oximeter applicator by Starr Life Sciences that is marketed for application to the tails of subjects (e.g. mice and small rodents), however, here the grooves 48 are sized to fit standard surgical tubing 24 (or sized to fit the tubing associated with the device). Other known techniques to maximize the transmitted and received signal of interest and to minimize the noise may be included, as desired.

The device 10 according to the present application has numerous uses, such as it may be used to rapidly and accurately form oxygen dissociation curves for given subjects, and other blood gas analysis, in given subjects, in the extracorporeal circuit shown. This use would serve both research and educational purposes. The blood gas analysis depends upon the particulars of the sensor 40, itself. For example, Massimo has developed sensors 40 that are acceptable for carbon monoxide measurements of blood. Most conventional pulse oximeter sensors 40 would be suitable for blood oxygen analysis.

It should be apparent that the conventional pulse oximeter sensors 40 will provide oxegenation, or other blood gas of interest, and a “pulse rate” measurement indicative of the pulsatile flow that is measured for Photoplethysmograph Pulse Oximetry Measurements. This “pulse rate” is related to the speed of the pump 30 and can be used to provide a feedback of the sensor 40 and/or the pump 30. If the measured value “pulse rate” from the sensor 40 does not match up with the designated speed of the pump 30 (measured with encoder or other speed control mechanism generally common on high end pumps), the device 10 can indicate an error (visually, audibly, or both, or other conventional error indication method).

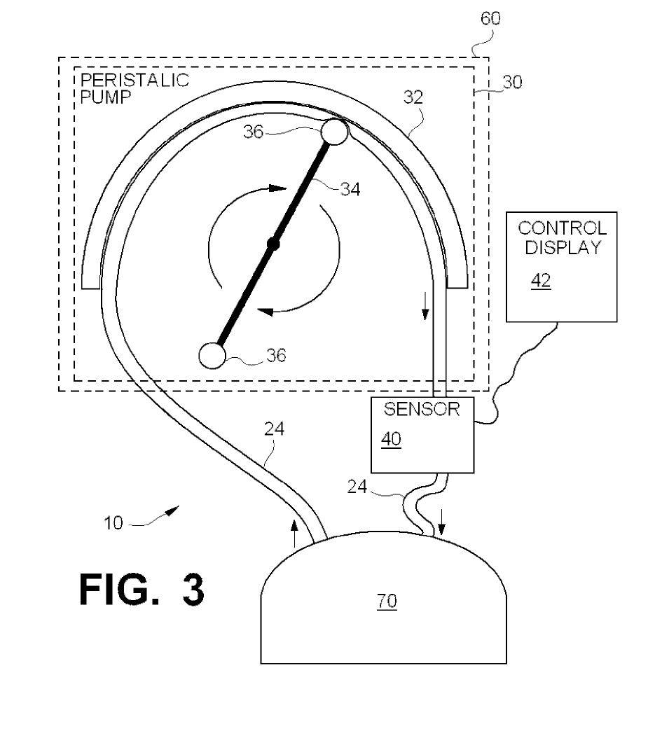

The device 10 is not intended to be limited to the extracorporeal circuit shown in FIG. 1, but can be used with any extracorporeal circuit that has a pulsatile flow and flexible transparent blood flow conduits. For example, FIG. 3 is an overview schematic view of a blood oxygenation monitoring device 10 according to the present invention used with an existing extracorporeal circuit 60 to precisely measure the oxygenation amounts of supplied blood. The source of the blood 14 is shown at 70 and may be a patient, a donor, or a reservoir system as shown in FIG. 1. The extracorporeal circuit 60 may be in the form of a conventional heart lung bypass machine, a machine for hemodialysis, a machine for hemofiltration, a machine for plasmapheresis, a machine for apheresis, or a machine for extracorporeal membrane oxygenation, or the like.

The requirements of the device 10, relevant to this initial embodiment of the present invention as described, is that the circuit 60 include a pulsatile pump 30 and at least partially transparent tubing 24, where Photoplethysmograph Pulse Oximetry Measurements are utilized. The requirements of the device 10, relevant to a second embodiment of the present invention as described further below, is that the circuit 60 include an at least partially transparent reservoir 12 to which photoplethysmographic sensors may be coupled and a pulse simulation mechanism configured to simulate pulsatile behavior of the blood within the reservoir relative to the photoplethysmographic sensors.

In these environments the sensor 40 will provide quick, reliable measurements of oxygenation (or other gas of interest measurement) of the blood being returned to the source 70 (e.g. the patient). This can be then compared with the measurements obtained from the patient themselves through, for example, a fingertip pulse oximeter. The patient measurements would be expected to have a certain lag time to them. Further, if the patient measurements were not tracking the leading measurements from the sensor 40, this can be an early indication of the onset of other problems that must be timely addressed by the caregivers.

As with FIG. 1, an alternative embodiment is the use of the sensor 40 with Photoplethysmograph Co-Oximetry Measurements. In this modification, flexible tubing is not required and no pulsetile behavior needs to be added to the blood. The flexible tubing may in fact be less desirable as the light attenuation of the tubing would more likely be easier to discount with solid (i.e. non-flexing) transparent tubing. The Photoplethysmograph Co-Oximetry Measurements will provide measurements for the gas of interest, but not feedback of pump speed as noted above in association with the Photoplethysmograph Pulse Oximetry Measurements based embodiment.

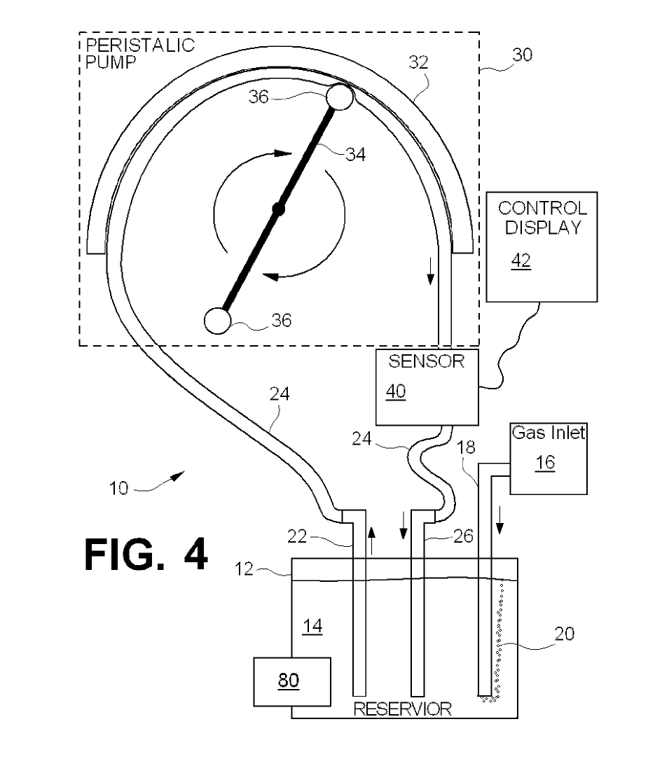

Another use of the device 10 according to the present invention is illustrated in FIG. 4 which is an overview schematic view of a blood oxygenation monitoring device 10 according to the present invention used for calibration of blood gas sensors. In this application the apparatus 10 is provided on or within a separate sensor 80, wherein the results of the sensor 40 are used to validate or calibrate those of sensor 80. The sensor 80 must be measuring the same gas as the sensor 40 but need not based upon photoplethsmography. It will be apparent from the following description of a second embodiment according to the present invention that the second embodiment may also be easily incorporated into the calibration system of FIG. 4.

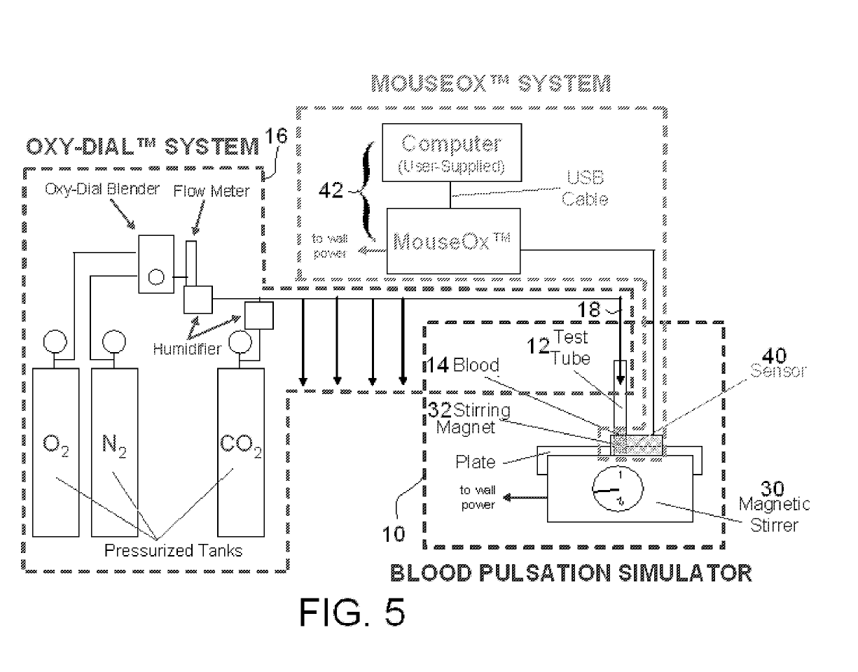

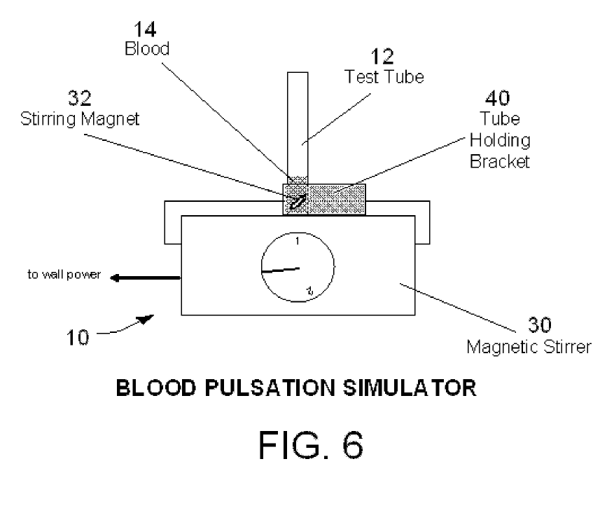

FIGS. 5 and 6 illustrate a device 10 according to a second embodiment of the present invention. The blood gas monitoring device 10 of FIGS. 5 and 6 still comprising an extracorporeal pulse simulation system wherein the extracorporeal pulse simulation system includes one at least partially transparent blood holding element. The transparent element is formed by the reservoir 12 instead of the tubing of the earlier embodiment. The photoplethysmographic sensor 40 is coupled to the blood holding element, namely reservoir 12, and adapted to measure particular gas content of the blood within the element. The device 10, in one embodiment, also includes an extracorporeal pulse simulation system includes a pulse simulation mechanism 30, formed by pump 30 in the initial embodiment and now formed by magnetic stirrer 30 and stir bar 32. The pulse simulation mechanism 30 is configured to simulate pulsatile behavior of the blood within the element (reservoir 12) relative to the photoplethysmographic sensors 40 as described below.

In the embodiment of FIGS. 5 and 6 the reservoir 12 can be and is preferably made very small, such as a standard glass or plastic test tube. Plastic test tubes have been found to have less detrimental effect on the light passing between the sensors than glass test tubes. Any transparent test tube material should work. The structure of this embodiment greatly reduces the priming volume of blood 14 needed for operation of the device 10. The device will operate with less than 10 cc of blood 14 within the reservoir 12, even less than 5 cc of blood, and it is expected that about 2 cc of blood will be sufficient for adequate operation. The structure of the device 10 allows for a minimal blood contact for setting up and implementing the device 10, which makes it advantageous for teaching environments, such as students learning about and conducting research and experiments relating to oxygen dissociation curves for various animals.

FIG. 5 also illustrates the device 10 used with an OXY-DIAL™ system forming the gas source 16 and coupling 18. The OXY-DIAL™ system is commercially available from Starr Life Sciences, Inc. and allows users, namely researchers, to easily and efficiently blend a series of gasses together to obtain desired ratios. The gasses shown in this embodiment are oxygen, nitrogen and carbon dioxide, but other gasses can be used as desired for the particular implementation. The gas source 16 is provided to allow the user to supply a selected gas, e.g. a 20% oxygen mixture, to the blood 14 as needed.

The sensor 40 in the embodiment of FIGS. 5 and 6 will be mounted on a structure that can also help support the reservoir 12, particularly if a test tube structure is used for the reservoir 12. A beaker or other convenient structure can be used for the reservoir 12, but the test tube is efficient, easily found and provided for small priming volume to the device 10.

FIG. 5 expressly illustrates that the sensor 40 is associated with a MouseOx™ brand pulse oximeter. This particular pulse oximeter does have the advantage of operating effectively using Photoplethysmograph Pulse Oximetry Measurements with a wider range of “pulse” ranges than other commercially available pulse oximeters making it well suited for use with the device 10, but other pulse oximeters could be utilized.

Without being limited to any particular theory of operation, the device 10 of FIGS. 5 and 6 may be designed to operate by having the stir bar 32 periodically interrupt the light path between the sensors 40. This rhythmic interruption of the sensor light path by the sir bar 32 may simulate pulsetile behavior of the blood within the reservoir 12 relative to the photoplethysmographic sensors 40. Effectively the variance of the light path will create the distinct measurements necessary for sensors 40 to obtain the desired measurements regarding blood oxygenation and the like using Photoplethysmograph Pulse Oximetry Measurements. Conventional sensors 40 using Photoplethysmograph Pulse Oximetry Measurements will return a “pulse” rate for the blood 14 which will be related to the speed, in revolutions per minute, of the stir bar 32. The speed of the stir bar 32 will be controlled by the magnetic stirrer 30 as known in the art of magnetic stirrers. Typically a control knob is rotated to increase the speed of the stir bar 32, wherein the actual rotational speed of the stir bar 32 will depend upon the viscosity of the blood 14 and the placement of the test tube reservoir 12 on the magnetic stirrer 30.

It is advantageous if the reservoir 12 has a rounded cross sectional shape, typically a circle is cross section. A square, rectangle or other shape could be used, but shapes that could have the stirrer stuck in the corners should be avoided. Further, the stirrer 32 may preferably be larger in a length direction than the diameter of the reservoir 12 to provide an angular position of the stir bar 12 within the tube or reservoir 12. This will allow a portion of the stir bar 32 to move completely into and out of the path of the light between the sensors 40 to better simulate a pulsatile action.

It may be advantageous if the reservoir 12 is placed off center on the top of the magnetic stirrer 30. Conventional stirrers 30 often have heating plates associated there with, and the device 10 of the present invention can also effectively use this device. A heater in the stirrer 30 can allow the user to set and maintain the temperature of the blood 14.

Other pulse simulation mechanisms could be utilized, such as a mechanical stirrer that has the stirring elements interfere with the light path in the same or similar manner as the stir bars 32 described above. However the ease of cleaning the mechanical stirrer 30 version is believed to offer significant advantages over a mechanical stirrer system. In such cleaning of the device of FIGS. 5 and 6, only the test tube or reservoir 12 and the stir bar 32 need be cleaned. In certain implementations, such as where blood contamination is a critical issue, these components can be disposed of without detrimentally affecting the overall costs. Test tubes 12 and stir bars 32 represent relatively inexpensive components. Further, blood 14 needs to be adequately contained when being disposed of, e.g. at the conclusion of an experiment, and keeping it in the test tube 12 for disposal with capping of tube (or not) and separate recovery (or not) of the stir bar 32 also being possible. Regarding the disposal features, plastic test tubes 12 (as opposed to glass) offer very inexpensive prospects for the present invention.

The embodiment of FIGS. 5 and 6 can be implemented using Photoplethysmograph Co-Oximetry Measurements for the sensors 40 and this yields certain advantages. In the embodiments using Photoplethysmograph Co-Oximetry Measurements the stir bar, if provided, need only be used to homogenize the blood, as is the more common function of the stir bar. The Photoplethysmograph Co-Oximetry Measurement based embodiments would not provide feedback relative to the speed of the stirrer as would the Photoplethysmograph Pulse Oximetry Measurement based embodiment described above.

The Photoplethysmograph Co-Oximetry Measurement based embodiments of the present invention, particularly of FIGS. 5 and 6, yield another embodiment of the present invention that does not lend itself to a Photoplethysmograph Pulse Oximetry Measurement based system. Namely if the transparent reservoir 12 were in the form of the body of a syringe and the Photoplethysmograph Co-Oximetry Measurement based sensor 40 were on the transparent reservoir 12/syringe body, then the system would allow for measurements of blood drawn directly from the subject. Further, following the obtaining of the desired measurements the blood can be returned to the subject through the syringe and associated needle. This syringe based system may be particularly advantageous for direct blood measurements of small subjects such as rats and mice that would not otherwise support repeated blood sample takings (without the intermediate return of the sampled blood).

In short the present invention provides a tool for clinicians, researchers, caregivers, educators and manufacturers that can be used in a number of distinct applications and although the present invention has been described with particularity herein, the scope of the present invention is not limited to the specific embodiment disclosed. It will be apparent to those of ordinary skill in the art that various modifications may be made to the present invention without departing from the spirit and scope thereof. For example, the sensors, or at least the active part of the sensors, could feasibly be incorporated directly into the wall of the tubing or flow conduit. The sensors/tubing could have a connector to which the sensor leads would be connected, or the leads could already be in place. Other modifications are also possible within the broad teaching of the present invention.

The scope of the invention is not to be limited by the illustrative examples described above. The scope of the present invention is defined by the appended claims and equivalents thereto.

What is claimed is:

1. A blood gas monitoring device comprising:
An at least partially transparent tubing having blood flow therethrough;
A pump coupled to the tubing and adapted to have blood flow therethrough;
A photoplethysmographic sensor coupled to the tubing and adapted to measure particular gas content of the blood within the tubing.

2. The blood gas monitoring device of claim 1 wherein the pump is a peristaltic pump forcing the blood to flow through the tubing in a pulsetile fashion.

3. The blood gas monitoring device of claim 2 wherein the photoplethysmographic sensor is a pulse oximeter adapted to measure oxygen within the blood in the tubing.

4. The blood gas monitoring device of claim 3 wherein the tubing is exiting a heart lung bypass machine, and wherein the pump is part of the heart lung bypass machine.

5. The blood gas monitoring device of claim 3 further including a reservoir coupled to the pump and a gas inlet for introducing gas into blood held in the reservoir.

6. The blood gas monitoring device of claim 3 wherein the coupling between the pulse oximeter and the tubing prevents ambient light from being received by the pulse oximeter.

7. A blood oxygenation monitoring device comprising:
An at least partially transparent tubing having blood flow therethrough;
A peristaltic pump coupled to the tubing and adapted to have blood flow therethrough;
A photoplethysmographic pulse oximeter sensor coupled to the tubing and adapted to measure oxygen content of the blood within the tubing.

8. The blood oxygenation monitoring device of claim 7 wherein the tubing is exiting a heart lung bypass machine, and wherein the pump is part of the heart lung bypass machine.

9. The blood oxygenation monitoring device of claim 7 wherein the coupling between the pulse oximeter and the tubing prevents ambient light from being received by the pulse oximeter.

10. A method of blood gas monitoring comprising the steps of:
Providing an at least partially transparent flexible tubing with a photoplethysmographic sensor coupled to the flexible tube;
Supplying blood flow through the tubing in a pulsetile manner;
measuring particular gas content of the blood within the tubing with the photoplethysmographic sensor.

11. The method of blood gas monitoring according to claim 10 further comprising the use of a peristaltic pump coupled to the tubing to create the pulsatile blood flow through the tubing.

12. The method of blood gas monitoring according to claim 11 wherein the flexible tubing is exiting a heart lung bypass machine, and wherein the pump is part of the heart lung bypass machine.

13. The method of blood gas monitoring according to claim 10 further including the step of preventing ambient light from being received by the photoplethysmographic sensor.

14. The method of blood gas monitoring according to claim 10 wherein the blood gas being monitored is the oxygenation of the blood.

15. The method of blood gas monitoring according to claim 14 further including repeating the steps to form an oxygen dissociation curve for the blood.

16. A blood gas monitoring device comprising:
An at least partially transparent blood holding reservoir;
A photoplethysmographic sensor coupled to the blood holding reservoir and adapted to measure particular gas content of the blood within the reservoir; and
A pulse simulation mechanism configured to simulate pulsatile behavior of the blood within the reservoir relative to the photoplethysmographic sensors.

17. The blood gas monitoring device according to claim 16 wherein the pulse simulation mechanism includes a magnetic stirrer and a stir bar within the reservoir.

18. The blood gas monitoring device according to claim 17 wherein the reservoir is a test tube.

19. A method of blood gas monitoring comprising the steps of:
Providing an at least at least partially transparent blood holding reservoir with a photoplethysmographic sensor coupled to the reservoir;
Supplying blood to the reservoir;
Simulating pulsatile behavior of the blood within the reservoir relative to the photoplethysmographic sensors;
measuring particular gas content of the blood within the reservoir with the photoplethysmographic sensor.

20. The method of blood gas monitoring according to claim 19 wherein the pulse simulation includes the use of a magnetic stirrer and a stir bar within the reservoir.

21. The method of blood gas monitoring according to claim 19 wherein the reservoir is a plastic test tube.

22. A blood gas monitoring device comprising an extracorporeal pulse simulation system wherein the extracorporeal pulse simulation system includes one at least partially transparent blood holding element with a photoplethysmographic sensor coupled to the blood holding element and adapted to measure particular gas content of the blood within the element, and the extracorporeal pulse simulation system includes a pulse simulation mechanism configured to simulate pulsatile behavior of the blood within the element relative to the photoplethysmographic sensors.

23. The blood gas monitoring device according to claim 22 wherein the blood holding element is a reservoir and wherein the pulse simulation mechanism includes a magnetic stirrer and a stir bar within the reservoir.

24. The blood gas monitoring device according to claim 22 wherein the extracorporeal pulse simulation system is an extracorporeal circuit and the blood holding element is an at least partially transparent flexible tubing having blood flow therethrough.

25. The blood gas monitoring device according to claim 22 wherein the pulse simulation mechanism is a peristaltic pump coupled to the tubing and adapted to have blood flow therethrough in a pulsatile manner.
